(19) United States
(12) Patent Application Publication
(10) Pub. No.: US 2003/0211595 A1
(43) Pub. Date: Nov. 13, 2003

(54) Rack for handling polymerase chain reaction tubes

(76) Inventor: Tzong Hae Lee, San Francisco, CA (US)

Correspondence Address: STEVENS DAVIS MILLER & MOSHER, LLP, 1615 L STREET, NW, SUITE 850, WASHINGTON, DC, 20036, US

(21) Appl. No.: 10/142,901

(22) Filed: May 13, 2002

(51) Int. Cl. C12M 1/00; A47G 29/00

(52) U.S. Cl. 435/283.1; 211/71.01; 422/104; 422/99; 422/102; 435/288.4; 211/74

(57) ABSTRACT

The present invention provides a rack for handling polymerase chain reaction (PCR) tubes. The rack includes a ceramic base and an array of receptacles formed therein. The ceramic base has opposing upper and lower surfaces. Each receptacle is configured to removably support one of the polymerase chain reaction tubes in a substantially upright fashion.

### BACKGROUND OF THE INVENTION

1. Field of the Invention

The present invention relates to a rack for handling tubes, and more particularly to a rack for handling polymerase chain reaction tubes.

2. Description of the Related Art

Polymerase chain reaction (PCR) is a rapid and simple method for specifically amplifying a target DNA sequence in an exponential manner. Briefly, the method as now commonly practiced utilizes a pair of primers that have nucleotide sequences complementary to the DNA which flanks the target sequence. The primers are mixed with a solution containing the target DNA (the template), a Taq DNA polymerase and dNTPs for all four deoxynucleotides (adenosine (A), tyrosine (T), cytosine (C) and guanine (G)). The mix is then heated to a temperature sufficient to separate the two complementary strands of DNA. The mix is next cooled to a temperature sufficient to allow the primers to specifically anneal to sequences flanking the gene or sequence of interest. The temperature of the reaction mixture is then set to the optimum for the thermophilic DNA polymerase to allow DNA synthesis (extension) to proceed. The temperature regimen is then repeated to constitute each amplification cycle. Thus, PCR consists of multiple cycles of DNA melting, annealing and extension. Twenty replication cycles can yield up to a million-fold amplification of the target DNA sequence.

Although significant progress has been made in PCR technology, the amplification of non-target oligonucleotides due to side-reactions, such as mispriming on non-target background DNA, RNA, and/or the primers themselves, still presents a significant problem. This is especially true in diagnostic applications where PCR is carried out in a milieu containing complex background DNA while the target DNA may be present in a single copy.

The temperature at which Taq DNA polymerase exhibits highest activity is in the range 62-72° C.; however, significant activity is exhibited at room temperature, approximately 25° C. to 37° C. Therefore, side reactions often occur when all reactants are mixed at ambient temperature before thermal cycling is initiated. The primers may prime DNA extension at non-specific sequences because the formation of only a few base pairs at the 3′-end of a primer can result in a stable priming complex. The result can be competitive or inhibitory products at the expense of the desired product. As an example of inhibitory product, structures consisting only of primer, sometimes called “primer dimers” are formed by the action of DNA polymerase on primers paired with each other, regardless of the true target template. The probability of undesirable primer-primer interactions increases with the number of primer pairs in the reaction, as with multiplex PCR. During PCR cycling, these non-specific extension products can compete with the desired target DNA.

One method for minimizing these side reactions is termed “hot start” PCR. Hot start PCR may be accomplished by various physical, chemical, or biochemical methods. In a physical hot start, the DNA polymerase or one or more reaction components that are essential for DNA polymerase activity is not allowed to contact the sample DNA until all the components required for the reaction are at a high temperature. Another way to implement a hot start PCR is to use DNA polymerase which is inactivated chemically but is reactivatable by heating. An additional way of implementing a hot start is to combine the Taq DNA polymerase enzyme with a Taq antibody before adding it to the reagent. This method employs a monoclonal, inactivating antibody raised against Taq DNA polymerase. The antibody inhibits the polymerase activity at ambient temperature but is inactivated by heat denaturation once the reaction is thermocycled, thus rendering the polymerase active.

However, current methods of minimizing aforementioned side reactions by achieving a hot start are tedious, expensive, and/or have other shortcomings. The present invention therefore seeks to provide a low tech, inexpensive option that overcomes, or at least reduces the above-mentioned problems of the prior art.

### SUMMARY OF THE INVENTION

It is an object of the present invention to provide a rack for holding PCR tubes and keeping the tubes and the reactants contained therein in a low temperature to minimize the chance of primer binding to the DNA template and to prevent the polymerase from working prior to the first heating step of PCR.

To achieve the above listed and other objects, the present invention provides a rack comprises a ceramic base and a support member. The support member has an array of apertures. It is preferred that the support member is made of plastic material. The ceramic base has an array of receptacles formed therein corresponding to the apertures of the support member. Each receptacle is configured to removably support one of the polymerase chain reaction tubes in a substantially upright fashion. The ceramic base has opposing upper and lower surfaces. It is preferred that the receptacle has two opening in both the upper surface and the lower surface of the ceramic base.

The ceramic base has a thermal conductivity within the range of from about 20 to about 150 W/m-K under 20° C., and more preferably, the thermal conductivity of the ceramic base is about 80 W/m-K under 20° C. If the rack is stored in a low temperature environment such as a refrigerator or a cooler in advance, when the rack is moved to the ambient temperature, the rack can still keep the low temperature for 30-40 minutes due to the low thermal conductivity of the ceramic base. The ceramic base is designed to be light-weight by keeping the density thereof within the range of from about 2.5 to about 5.5 g/cm³.

The present invention provides a light, and a simple rack to hold the PCR tubes and keep them below a predetermined temperature such as 4° C. if the rack is stored in a refrigerator or a cooler in advance.

### BRIEF DESCRIPTION OF THE DRAWINGS

Other objects, advantages, and novel features of the invention will become more apparent from the following detailed description when taken in conjunction with the accompanying drawings:

FIG. 1 is a perspective view illustrating a supporting member and a base of a rack according to one embodiment of the present invention;

FIG. 2A is a top elevational view of the base shown in FIG. 1;

FIG. 2B is a bottom elevational view of the base shown in FIG. 1;

FIG. 3 is a perspective view of a conventional multi-tube plate used in PCR; and

FIG. 4 is a perspective view of a centrifuge holding the rack shown in FIG. 1 with the multi-tube plate shown in FIG. 3.

### DETAILED DESCRIPTION OF THE PREFERRED EMBODIMENT

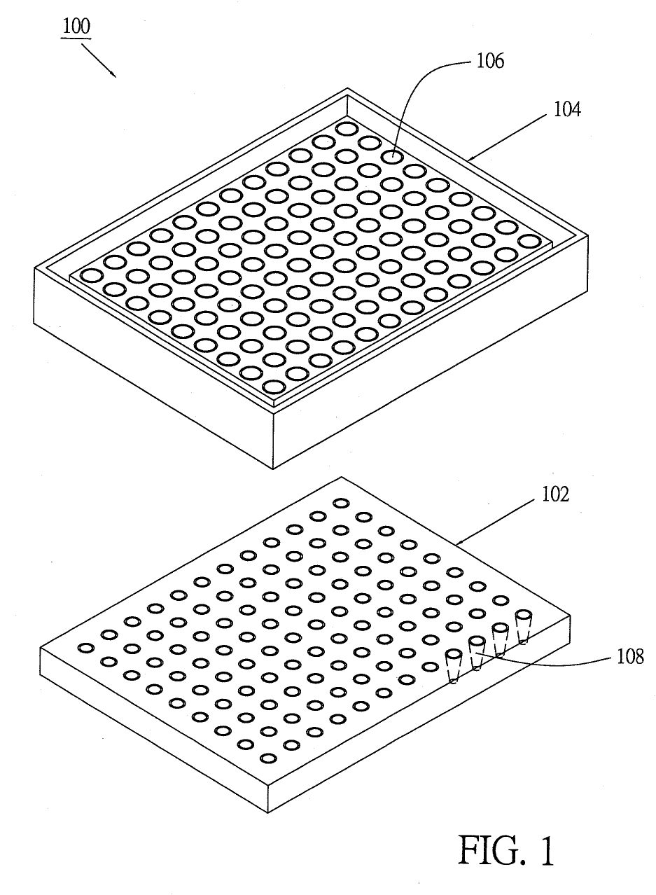

FIG. 1 is a perspective view illustrating a rack 100 according to one embodiment of the present invention. The rack 100 mainly includes a ceramic base 102 and a support member 104. The support member 104 has an array of apertures (only one designated by reference numeral 106). The ceramic base 102 includes an array of receptacles (only one designated by reference numeral 108) formed therein. The support member 104 is provided on the ceramic base 102 in a manner that each receptacle 108 of the ceramic base is aligned with one aperture 106 of the support member 104. Each of the receptacles 108 is configured to receive one tube used in polymerase chain reaction (PCR) in a substantially upright fashion. When the support member 104 is provided on the ceramic base 102, the PCR tubes can be put into the receptacles 108 through the apertures 106 and surrounded by the ceramic material. Alternatively, the ceramic base 102 can be utilized to receive the PCR tubes alone. The ceramic base has a thickness at least sufficient to surround the reaction solution contained in the tubes held therein. The thermal conductivity of the ceramic material utilized in the present invention is preferably kept within the range of from about 20 to about 150 W/m-K under 20° C. It is preferred that the ceramic material has a thermal conductivity about 80 W/m-K under 20° C. Before operation, the rack 100 can be stored under a low temperature, e.g., under 0° C., in a freezer or a refrigerator. Thereafter, the ceramic base can stay cold for a predetermined time period, e.g., 20-30 minutes, for preparing and mixing the reactants of PCR.

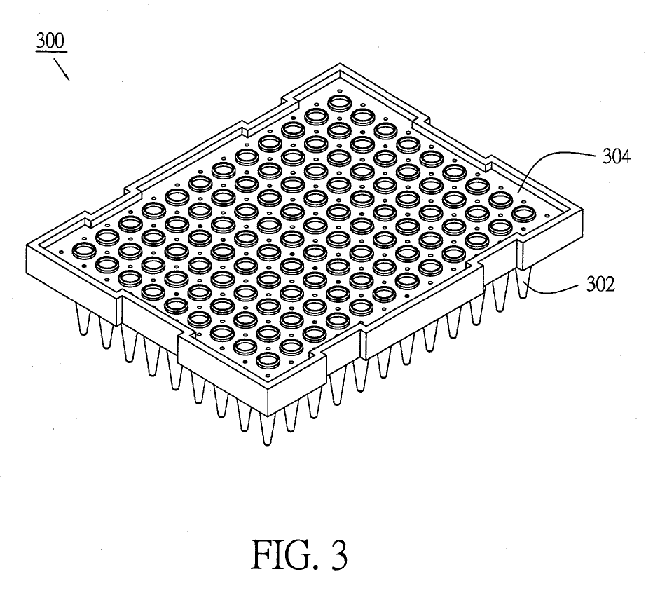

Typically, it is desirable to integrate a plurality of PCR tubes in a array configuration to form a multi-tube plate 300 (see FIG. 3). The multi-tube plate 300 comprises a substantially flat member 304 surrounding the opening of the tubes 302 and connecting each of the tubes 302.

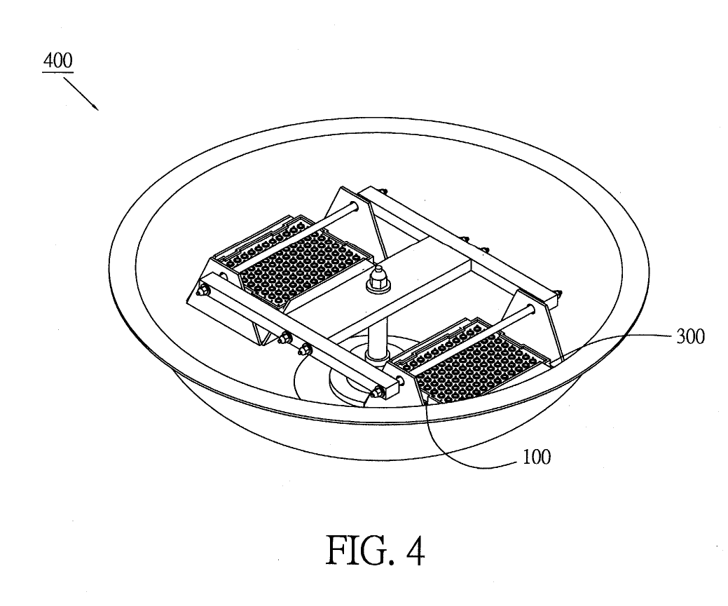

Next, a method for manufacturing a rack according to one embodiment of the present invention will be described. First, a support member 104 having a wall erected around a space adapted for receiving the aformentioned multi-tube plate 300 or the like is provided. It is preferred that the support member is made of plastic material. Then, a multi-tube plate or the like is placed on the support member with the tubes disposed in the apertures of the support member. Preferably, the multi-tube plate is removably attached to the support member. Thereafter, a ceramic material is dispensed onto the support member and the tubes of the multi-tube plate exposed through the apertures of the support member. After the ceramic material is cured, the multi-tube plate is removed thereby obtaining a ceramic base with an array of receptacles each shaped generally to conform to the shape of the tube. In setting up a PCR reaction, the aforementioned multi-tube plate 300 is disposed in the pre-cooled rack, and then the reactants are pipetted in each of the tubes. However, during the pipetting, some reactant may remain on the wall of the tube. Therefore, a centrifugal operation is necessary for collecting the reactants on the wall of the tube in the bottom thereof. In order to keep the tubes and the reactants therein under a constant low temperature environment, it is preferred that the multi-tube plate 300 is disposed in the rack of the present invention during the centrifugal operation (see FIG. 4). Therefore, the ceramic base is designed to be light-weight by keeping the density thereof within the range of from about 2.5 to about 5.5 g/cm3.

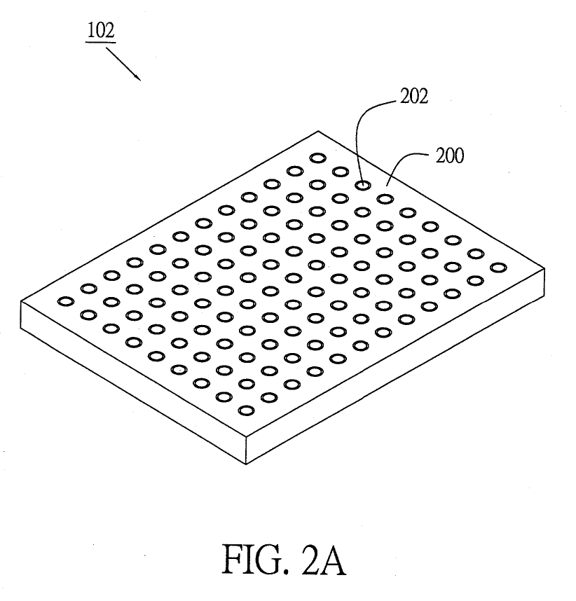

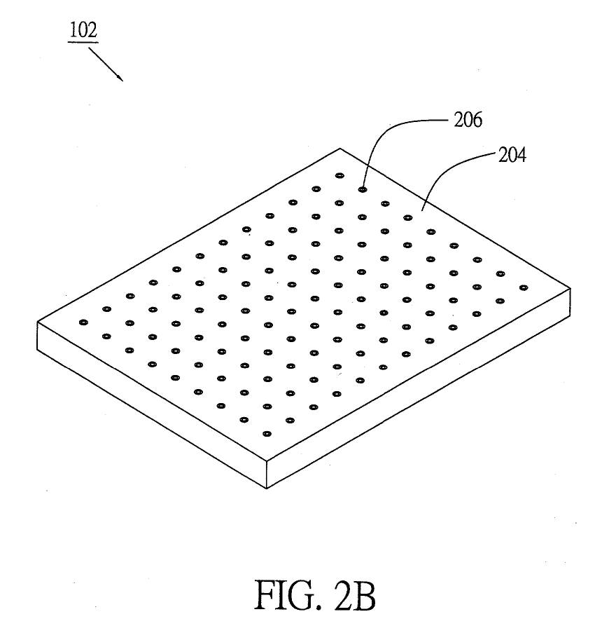

Steam sterilization (autoclave) is commonly adopted for sterilizing the tubes and other equipments (including the rack) used in PCR operation. Steam sterilization is a time proven and economical process of killing microorganisms through the application of moist heat (saturated steam) under pressure. Heat damages the cell's essential structures including the cytoplasmic membrane rendering the cell no longer viable. The rate by which bacterial cells are thermally inactivated depends on the temperature and the time of heat exposure to which they are exposed. However, water condensed from moisture in the steam tend to remain in go the receptacles of the ceramic base of the rack. Therefore, the receptacles of the ceramic base preferably have two openings defined in the opposing upper and lower surfaces of the ceramic base. Referring to FIG. 2A, the receptacle 108 has an opening 202 defined in the upper surface 200 of the ceramic base 102. As shown in FIG. 2B, the receptacle 108 also has an opening 206 defined in the lower surface 204 of the ceramic base 102. The present invention provides a light, and a simple rack to hold the PCR tubes and keep them below a predetermined temperature such as 4° C. if the rack is stored in a refrigerator or a cooler in advance. The rack of the present invention provides a steadily low temperature environment for preparing and mixing the reactants of PCR thereby minimizing the chance of primer binding to the DNA template and preventing the polymerase from working prior to the first heating step of PCR.

Although the invention has been explained in relation to its preferred embodiment, it is to be understood that many other possible variations can be made without departing from the spirit and scope of the invention as hereinafter claimed.

What is claimed is:

1. A rack for handling polymerase chain reaction (PCR) tubes comprising:
a ceramic base having opposing upper and lower surfaces; and
an array of receptacles formed in the ceramic base, each receptacle being configured to removably support one of the polymerase chain reaction tubes in a substantially upright fashion.

2. The rack as claimed in claim 1, wherein each of the receptacles has a first opening defined in the upper surface of the ceramic base adapted for receiving the tube, and at least one receptacle further has a second opening defined in the lower surface of the ceramic base.

3. The rack as claimed in claim 1, wherein the ceramic base has a density within the range of from about 2.5 to about 5.5 g/cm³.

4. The rack as claimed in claim 1, wherein the ceramic base has a thermal conductivity within the range of from about 20 to about 150 W/m-K under 20° C.

5. The rack as claimed in claim 4, wherein the ceramic base has a thermal conductivity about 80 W/m-K under 20° C.

6. A rack for handling a multi-tube plate used in polymerase chain reaction (PCR), the rack comprising:
a ceramic base having opposing upper and lower surfaces;
a support member provided on the ceramic base for holding the multi-tube plate, the support member having an array of apertures; and
an array of receptacles formed in the ceramic base,
wherein the apertures of the support member and the corresponding receptacles of the ceramic base are configured to removably support the tubes in a substantially upright fashion.

7. The rack as claimed in claim 6, where in the moveable support member is made of plastic material.

8. The rack as claimed in claim 6, wherein each of the receptacles has a first opening defined in the upper surface of the ceramic base adapted for receiving the tube, and at least one receptacle further has a second opening defined in the lower surface of the ceramic base.

9. The rack as claimed in claim 6, wherein the ceramic base has a density within the range of from about 2.5 to about 5.5 g/cm³.

10. The rack as claimed in claim 6, wherein the ceramic base has a thermal conductivity within the range of from about 20 to about 150 W/m-K under 20° C.

11. The rack as claimed in claim 10, wherein the ceramic base has a thermal conductivity about 80 W/m-K under 20° C.
